(19) United States
(12) Patent Application Publication
(10) Pub. No.: US 2015/0173627 A1
(43) Pub. Date: Jun. 25, 2015

(54) PULSE MEASUREMENT DEVICE, PULSE MEASUREMENT METHOD, AND PULSE MEASUREMENT PROGRAM

(71) Applicant: OMRON HEALTHCARE CO., LTD., Muko-shi (JP)

(72) Inventors: Kenji FUJII, Kyoto (JP); Tatsuya KOBAYASHI, Kyoto (JP); Toshihiko OGURA, Kyoto (JP); Yukiya SAWANOI, Kyoto (JP)

(21) Appl. No.: 14/641,546

(22) Filed: Mar. 9, 2015

Related U.S. Application Data
(63) Continuation of application No. PCT/JP2013/070750, filed on Jul. 31, 2013.

(30) Foreign Application Priority Data
Sep. 13, 2012 (JP) 2012-201910

(51) Int. Cl. A61B 5/024; A61B 5/00

(52) U.S. Cl. CPC A61B 5/024; A61B 5/7207; A61B 5/7235; A61B 5/681

(57) ABSTRACT

A pulse measurement device according to an aspect of the present invention includes a data obtainment unit that obtains a pulse wave signal by detecting a pulse wave using a pulse wave sensor, an exercise intensity obtainment unit that obtains an exercise intensity signal by detecting movement using a body movement sensor, a storage unit that stores the pulse wave signal, a frequency conversion unit that finds a frequency spectrum of the pulse wave signal by converting the time-domain pulse wave signal into a frequency domain, a searched range setting unit that sets a searched frequency range for searching for an intensity peak along a frequency axis of the frequency spectrum, a peak extraction unit that extracts an intensity peak from the searched frequency range, and a pulse rate calculation unit that finds a pulse rate of the measurement subject based on a frequency of the extracted intensity peak. The searched range setting unit changes the searched frequency range based on an exercise intensity indicated by the exercise intensity signal.

### BACKGROUND OF THE INVENTION

1. Field of the Invention

This invention relates to pulse measurement devices, and particularly relates to pulse measurement devices that measure a pulse rate by detecting pulsatory motion in a blood vessel of a measurement subject.

This invention also relates to pulse measurement methods and pulse measurement programs, and particularly relates to pulse measurement methods and pulse measurement programs for measuring a pulse rate by detecting pulsatory motion in a blood vessel of a measurement subject.

2. Description of the Related Art

A device that measures a measurement subject's pulse rate (heart rate) by wrapping a belt to which an electrocardiographic sensor is attached around the measurement subject's chest area and measuring the beating of the measurement subject's heart electrocardiographically can be given as a conventional device for measuring a measurement subject's pulse.

There is also a device that measures a pulse rate by detecting pulsatory motion in a measurement subject's blood vessel in a non-electrocardiographic manner, unlike the aforementioned device that electrocardiographically detects a measurement subject's heartbeat.

A device that measures a measurement subject's pulse rate by photoelectrically detecting pulsatory motion in a measurement subject's subcutaneous blood vessel using a photoelectric sensor can be given as an example of the latter type of device (see JP H10-234684A), for example).

In the latter type of device, a signal expressing pulsatory motion in the measurement subject's subcutaneous blood vessel (a pulse wave signal) is obtained and the pulse rate is measured based on the cyclic nature of fluctuations in the pulse wave signal over time.

### SUMMARY OF THE INVENTION

However, with a device that employs a method in which the measurement subject's pulse rate is measured by detecting pulsatory motion in the measurement subject's subcutaneous blood vessel non-electrocardiographically, such as photoelectrically, it is difficult to correctly measure the measurement subject's pulse rate when the measurement subject is exercising, for example.

The reason for this is that if the measurement subject is exercising during the measurement, the blood vessel experiences acceleration due to the exercise, and irregularities arise in the blood flow as a result. These irregularities are superimposed on the pulse wave signal as external disturbance components. This makes it difficult to extract the cycle of the temporal fluctuations caused by the pulsatory motion from the pulse wave signal.

Meanwhile, when the measurement subject is exercising, a sensor means attached to a part of the measurement subject's body will also experience acceleration, which results in a phenomenon in which the sensor means shifts position relative to that part of the body, separates from the part of the body even temporarily, and so on. This phenomenon also appears as an external disturbance component superimposed on the pulse wave signal. Such a phenomenon is another cause of difficulty in extracting the cycle of the temporal fluctuation caused by the pulsatory motion from the pulse wave signal.

In the pulse wave signal, it is extremely difficult to differentiate between fluctuations in the signal strength caused by pulsatory motion in the blood vessel and fluctuations in the signal strength caused by external disturbance components as mentioned above. Accordingly, in the case where a method for measuring a measurement subject's pulse rate by detecting pulsatory motion in the measurement subject's subcutaneous blood vessel non-electrocardiographically, such as photoelectrically, is employed, it has been necessary for the measurement subject to remain at rest during the measurement in order to prevent the aforementioned external disturbance component from being superimposed on the pulse wave signal.

This has limited the usability of the pulse measurement device, the diversity of applicable measurement conditions and measurement environments, and so on.

Accordingly, it is an advantage of this invention to provide a pulse measurement device capable of correctly measuring a measurement subject's pulse rate even in the case where the measurement subject is not at rest.

It is a further advantage of this invention to provide a pulse measurement method capable of correctly measuring a measurement subject's pulse rate even in the case where the measurement subject is not at rest, and a pulse measurement program capable of causing a computer to execute such a pulse measurement method.

A pulse measurement device according to an aspect of this invention includes a data obtainment unit configured to obtain a pulse wave signal expressing a pulse by detecting a pulse wave of a measurement subject using a pulse wave sensor, an exercise intensity obtainment unit configured to obtain an exercise intensity signal expressing an intensity of exercise performed by the measurement subject by detecting movement in the measurement subject using a body movement sensor, a storage unit configured to store the pulse wave signal, a frequency conversion unit configured to find a frequency spectrum of the pulse wave signal by converting the time-domain pulse wave signal stored in the storage unit into a frequency domain, a searched range setting unit configured to set a searched frequency range for searching for an intensity peak along a frequency axis of the frequency spectrum, a peak extraction unit configured to extract an intensity peak from the frequency spectrum in the set searched frequency range, and a pulse rate calculation unit configured to find a pulse rate of the measurement subject based on a frequency of the extracted intensity peak; the searched range setting unit changes the searched frequency range based on an exercise intensity indicated by the exercise intensity signal.

Note that in the present specification, the data obtainment unit may obtain the pulse wave signal directly from the pulse wave sensor, or may instead temporarily store the pulse wave signal from the pulse wave sensor in a server (having a storage unit) and then obtain (indirectly obtain) the signal from the server or the like. Furthermore, the exercise intensity obtainment unit may obtain the exercise intensity signal directly from the body movement sensor, or may instead temporarily store the exercise intensity signal from the body movement sensor in a server (having a storage unit) and then obtain (indirectly obtain) the signal from the server or the like.

Here, “pulse rate” refers to a number of pulses per unit of time (for example, beats per minute (BPM), which is the number of pulses per minute).

In the pulse measurement device according to this aspect of the invention, the data obtainment unit obtains the pulse wave signal expressing the pulse by detecting the pulse wave of the measurement subject using the pulse wave sensor. The exercise intensity obtainment unit obtains the exercise intensity signal expressing the intensity of exercise performed by the measurement subject by detecting movement in the measurement subject using the body movement sensor. The storage unit stores the pulse wave signal. The frequency conversion unit finds a frequency spectrum of the pulse wave signal by converting the time-domain pulse wave signal stored in the storage unit into the frequency domain. The searched range setting unit sets the searched frequency range for searching for the intensity peak along the frequency axis of the frequency spectrum. The peak extraction unit extracts the intensity peak from the frequency spectrum in the set searched frequency range. The pulse rate calculation unit finds the pulse rate of the measurement subject based on the frequency at the extracted intensity peak. Then, the searched range setting unit changes the searched frequency range based on the exercise intensity indicated by the exercise intensity signal.

Here, the searched range setting unit setting the searched frequency range for searching for the intensity peak along the frequency axis of the frequency spectrum means removing, from the frequency range in which the peak extraction unit extracts the intensity peak, a frequency component and harmonic components produced by the measurement subject's exercise. The measurement subject's exercise also affects his or her own pulse. For example, the measurement subject's pulse rate tends to increase when the measurement subject exercises vigorously. The measurement subject's pulse rate also tends to drop when the measurement subject reduces the intensity of his or her exercise. Accordingly, by predicting a trend in fluctuations in the pulse rate based on the exercise intensity indicated by the exercise intensity signal and changing the searched frequency range, it can be ensured that of the pulse wave signal, (a basic frequency component of) the frequency component produced by pulsatory motion in the measurement subject's blood vessel is present in the searched frequency range. Therefore, the measurement subject's pulse rate can be correctly calculated even when the measurement subject is not at rest.

In the pulse measurement device according to a preferred embodiment, the frequency conversion unit, the exercise intensity obtainment unit, the searched range setting unit, the peak extraction unit, and the pulse rate calculation unit repeat the aforementioned process at a predetermined cycle, and in the case where the pulse rate calculation unit has calculated a first value as the pulse rate of the measurement subject in a first cycle, the searched range setting unit sets a value present in a predetermined ratio range relative to the first value as the searched frequency range for a second cycle that follows the first cycle.

In the pulse measurement device according to this preferred embodiment, in the case where the first value has been calculated as the measurement subject's pulse rate in the first cycle, a value present in the predetermined ratio range relative to the first value is set as the searched frequency range for the second cycle that follows the first cycle. Accordingly, it is certain that (a basic frequency component of) the frequency component produced by pulsatory motion in the measurement subject's blood vessel in the searched frequency range is present in the searched frequency range in the second cycle as well. Therefore, the measurement subject's pulse rate can be correctly calculated even when the measurement subject is not at rest.

In the pulse measurement device according to a preferred embodiment, in the case where an exercise intensity obtained by the exercise intensity obtainment unit in a fourth cycle that follows a third cycle is greater than an exercise intensity obtained in the third cycle, the searched range setting unit sets a frequency range shifted toward a higher frequency than the searched frequency range for the third cycle as the searched frequency range for the fourth cycle.

In the pulse measurement device according to this preferred embodiment, in the case where the exercise intensity obtained in the fourth cycle is greater than the exercise intensity obtained in the third cycle, a frequency range shifted toward a higher frequency than the searched frequency range for the third cycle as the searched frequency range for the fourth cycle. By doing so, it is more certain that (a basic frequency component of) the frequency component produced by pulsatory motion in the measurement subject's blood vessel in the searched frequency range is present in the searched frequency range in the fourth cycle as well. Accordingly, the pulse rate can be calculated correctly even in the case where the measurement subject's exercise intensity has changed.

In the pulse measurement device according to a preferred embodiment, the searched range setting unit sets the searched frequency range for the fourth cycle so as to have the same spectral space as the searched frequency range for the third cycle.

Note that the “spectral space of the searched frequency range” in the present specification refers to an absolute value of the difference between a frequency corresponding to an upper limit of the searched frequency range and a frequency corresponding to a lower limit of the searched frequency range. The unit for this frequency may be BPM or the like.

In the pulse measurement device according to this preferred embodiment, the burden of processing performed by the device can be lightened.

A pulse rate measurement method according to another aspect of the invention is a method for measuring a pulse rate of a measurement subject carried out by a pulse measurement device, and includes a data obtainment step of obtaining a pulse wave signal expressing a pulse of the measurement subject using a pulse wave sensor, a storage step of storing the pulse wave signal in a storage unit, a frequency conversion step of finding a frequency spectrum of the pulse wave signal by converting the time-domain pulse wave signal stored in the storage unit into a frequency domain, an exercise intensity obtainment step of obtaining an exercise intensity signal expressing an intensity of exercise performed by the measurement subject using a body movement sensor, a searched range setting step of setting a searched frequency range for searching for an intensity peak along a frequency axis of the frequency spectrum, a peak extraction step of extracting an intensity peak from the frequency spectrum in the set searched frequency range, and a pulse rate calculation step of finding the pulse rate of the measurement subject based on a frequency of the extracted intensity peak; the searched range setting step includes a step of changing the searched frequency range based on an exercise intensity indicated by the exercise intensity signal.

According to the pulse rate measurement method according to this other aspect of the invention, by changing the searched frequency range based on the exercise intensity indicated by the exercise intensity signal, it can be ensured that of the pulse wave signal, (a basic frequency component of) the frequency component produced by pulsatory motion in the measurement subject's blood vessel is present in the searched frequency range. Therefore, the measurement subject's pulse rate can be correctly calculated even when the measurement subject is not at rest.

A pulse rate measurement computer program according to still another aspect of the invention is a program for causing a computer to execute the aforementioned pulse rate measurement method.

According to the pulse rate measurement computer program according to this other aspect of the invention, a computer can be caused to execute the aforementioned pulse measurement method.

As is clear from the foregoing, according to the pulse measurement device and the pulse rate measurement method according to these aspects of the invention, by changing the searched frequency range based on the exercise intensity indicated by the exercise intensity signal, it can be ensured that of the pulse wave signal, (a basic frequency component of) the frequency component produced by pulsatory motion in the measurement subject's blood vessel is present in the searched frequency range. Therefore, the measurement subject's pulse rate can be correctly calculated even when the measurement subject is not at rest.

Furthermore, according to the pulse rate measurement computer program according to an aspect of the invention, a computer can be caused to execute the aforementioned pulse rate measurement method.

The above and other elements, features, steps, characteristics and advantages of the present invention will become more apparent from the following detailed description of the preferred embodiments with reference to the attached drawings.

### BRIEF DESCRIPTION OF THE DRAWINGS

FIG. 1A is a schematic perspective view of the exterior of a pulse measurement device according to a preferred embodiment of this invention.

FIG. 1B is a schematic cross-sectional view of the pulse measurement device according to a preferred embodiment of this invention.

FIG. 2 is a block diagram illustrating the functional configuration of the pulse measurement device.

FIG. 3 is a diagram illustrating an example of the circuit configuration of a pulse wave sensor unit for measuring a pulse wave signal in the pulse measurement device.

FIG. 4 is a diagram illustrating a flow of operations performed by the pulse measurement device.

FIG. 5A is a diagram illustrating an example of a pulse wave signal (a time domain).

FIG. 5B is a diagram illustrating an example of an AC component in a pulse wave signal (a time domain).

FIG. 6 is a diagram illustrating an example of a pulse wave signal AC component (a frequency domain).

FIG. 7(a) is a diagram illustrating an example of temporal changes in the exercise intensity of a measurement subject, and FIG. 7(b) is a diagram illustrating a relationship between a pulse rate calculation timing and a time range of a pulse wave signal AC component used in the calculation of the pulse rate at each timing.

FIG. 8 is a diagram illustrating an example of a searched frequency range that is changed based on the exercise intensity.

### DETAILED DESCRIPTION OF THE PREFERRED EMBODIMENTS

Hereinafter, a preferred embodiment of the invention will be described in detail with reference to the drawings.

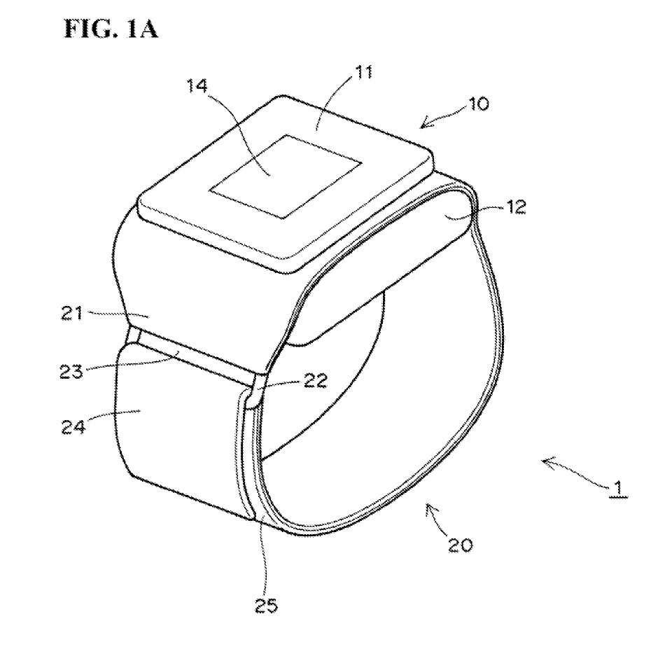

FIGS. 1A and 1B schematically illustrate the configuration of a pulse measurement device according to a preferred embodiment. FIG. 1A is a schematic perspective view of the exterior of the pulse measurement device according to the preferred embodiment, and FIG. 1B is a schematic cross-sectional view of the same pulse measurement device. Note that for descriptive purposes, a side of a main body 10 located toward a measurement area (not shown) will be referred to as a “bottom surface side”, whereas a side of the main body 10 on the side opposite from the measurement area will be referred to as a “top surface side”.

A pulse measurement device 1 includes the main body 10 and a band 20. As illustrated in FIGS. 1A and 1B, by wrapping the band 20 around a measurement area 3 (the wrist, for example) of a measurement subject like a wristwatch, the pulse measurement device 1 can affix the main body to the measurement subject's wrist.

The main body 10 of the pulse measurement device 1 includes a bottom surface 13 that is disposed in tight contact with the measurement area 3 of the measurement subject and forms a surface of contact with the measurement area, and a top surface 11 located on the side opposite from the bottom surface 13. The main body 10 has a recessed shape w in which the size of the main body 10 is configured to be smaller in a planar direction that follows the bottom surface 13 (see FIG. 1B).

The main body 10 of the pulse measurement device 1 includes a measurement unit 15 that is disposed on the bottom surface 13 side and is configured of a pulse wave sensor that measures the measurement subject's pulse, and a display unit 14 that is disposed on the top surface 11 side and displays information regarding the pulse measured by the measurement unit 15. The measurement unit 15 disposed on the bottom surface 13 side is an optical sensor that includes a light-emitting element 16, such as a light-emitting diode, that emits measurement light (infrared light or near-infrared light, for example), and a light-receiving element 17 such as a photodiode or a phototransistor. The light-emitting element 16 functions as a light-emitting unit that irradiates the measurement area with light having a given emitted light intensity. Meanwhile, the light-receiving element 17 functions as a light-receiving unit that receives reflected light or transmitted light from the measurement area.

When the main body 10 is disposed in tight contact with the measurement area 3 and a subcutaneous blood vessel (an artery, for example) in the measurement area is irradiated with the measurement light (infrared light or near-infrared light, for example) emitted from the light-emitting element 16, the irradiated light is reflected by red blood cells flowing in the artery and the reflected light is received by the light-receiving element 17. The amount of reflected light received by the light-receiving element 17 changes depending on pulsatory motion in the artery. Accordingly, pulse wave information can be detected and the pulse rate can be measured by the measurement unit 15. Although the measurement unit 15 is disposed so as to make contact with the bottom surface 13 in FIG. 1B, it should be noted that the configuration may be such that the measurement unit 15 is disposed within the main body 10 and a spatial portion that communicates between the measurement unit 15 disposed within the main body 10 and the bottom surface 13 of the main body 10 is provided. Furthermore, although the pulse measurement device 1 illustrated in FIGS. 1A and 1B is a type in which the measurement unit 15 is configured of the light-emitting element 16 and the light-receiving element 17 disposed in the vicinity of the light-emitting element 16 and detects light reflected by the measurement area 3, the device may be a type in which the measurement unit 15 is configured of the light-emitting element 16 and the light-receiving element 17 disposed facing the light-emitting element 16 and detects transmitted light that has passed through the measurement area 3.

The pulse measurement device 1 includes the measurement unit 15, configured of a photoelectric sensor, as a pulse wave sensor, and thus the pulse wave information, including the pulse, can be detected accurately with a simple configuration.

The display unit 14 is disposed on the top surface 11 side, or in other words, in a top area of the main body 10. The display unit 14 includes a display screen (for example, a liquid-crystal display (LCD) or an electroluminescence (EL) display). The display unit 14 displays information regarding the measurement subject's pulse (the pulse rate, for example) and so on in the display screen. Control of the display screen is carried out by a control unit 31 (a CPU) (mentioned later) functioning as a display control unit.

The band 20 for affixing the main body 10 to the measurement area 3 of the measurement subject includes a main body holding portion 21 for holding the main body 10 in tight contact and a wrapping portion 25 for wrapping around the measurement area.

An opening is formed in the main body holding portion 21 so as to approximately match the outer size of the recessed shape w in the main body 10, and the main body 10 is engaged with the band 20 in the area corresponding to the recessed shape w.

A buckle member 22 that is bent into an approximately rectangular shape is attached to one end of the main body holding portion 21. An end portion 24 of the wrapping portion 25 is passed through a hole 23 in the buckle member 22 so as to face outward from the measurement area 3, and is then folded back.

A relatively long female-side surface fastener that extends in a longer direction is provided on an outside surface (a surface opposite from an inside surface that makes contact with the measurement area 3) in an area of the wrapping portion 25 aside from the end portion 24, and the female-side surface fastener engages in a removable manner with a male-side surface fastener 26 that is attached to the end portion 24.

The main body 10 is held in tight contact with the measurement area 3 by the band 20 in this manner.

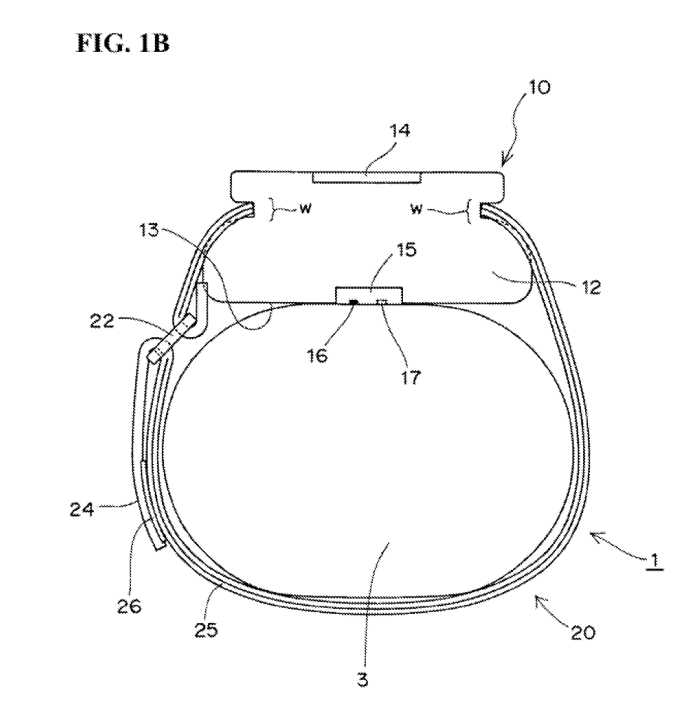

FIG. 2 illustrates a functional block configuration of the pulse measurement device 1. The main body 10 of the pulse measurement device 1 includes the control unit (CPU) 31, a storage unit 32, the display unit 14, an operating unit 34, the pulse wave sensor unit 15, and a body movement sensor unit 33. The pulse measurement device 1 may further include a communication unit (not shown). In this case, the pulse measurement device 1 can carry out data communication with an external device (not shown).

The control unit 31 includes a central processing unit (CPU) as well as auxiliary circuitry thereof, controls the various units that configure the pulse measurement device 1, and executes various types of processes in accordance with programs and data stored in the storage unit 32. In other words, the control unit (CPU) 31 processes data inputted from the operating unit 34, the pulse wave sensor unit 15, the body movement sensor unit 33, and the communication unit (not shown), and stores the processed data in the storage unit 32, displays the processed data in the display unit 14, outputs the processed data from the communication unit, and so on.

The storage unit 32 includes a RAM (random access memory) used as a work region required by the control unit (CPU) 31 to execute programs, and a ROM (read-only memory) for storing basic programs to be executed by the control unit (CPU) 31. A semiconductor memory (a memory card, a solid-state drive (SSD)) or the like may be used as a storage medium in an auxiliary storage unit for complementing a storage region in the storage unit 32. The storage unit 32 can store, in time series, the pulse wave signal (and an AC component thereof in particular) expressing the measurement subject's pulse as detected by the pulse wave sensor unit 15, on a measurement subject-by-measurement subject basis.

The operating unit 34 includes, for example, a power switch manipulated to turn the pulse measurement device 1 on or off, and an operating switch manipulated to select the measurement subject for whom a measurement result obtained on a measurement subject-by-measurement subject basis is to be saved in the storage unit 32 or to select the type of measurement to be carried out. Note that the operating unit 34 can be provided on the top surface 11 of the main body 10 (see FIG. 1A) or a side surface 12 (FIG. 1A).

In this manner, the pulse measurement device 1 can be configured as an independent device. However, providing the communication unit (not shown) makes it possible to use the device on a network as well.

The communication unit is used in order to send data generated by the control unit (CPU) 31, data stored in the storage unit 32, and so on to a server over a wired or wireless network, to receive data generated by a control unit (not shown) of the server, data stored in a storage unit (not shown) of the server, and so on, and the like. Here, “server” is a broad concept that includes, for example, a stationary terminal such as a personal computer, a mobile terminal such as a cellular phone, a smartphone, a PDA (personal digital assistant), a tablet, or a remote controller for an AV device such as a television, as well as a computer provided in an AV device such as a television, in addition to a normal server.

Note that power is supplied from a power source (not shown) to the various units in the pulse measurement device 1 in response to a user operation made through the power switch of the operating unit 34.

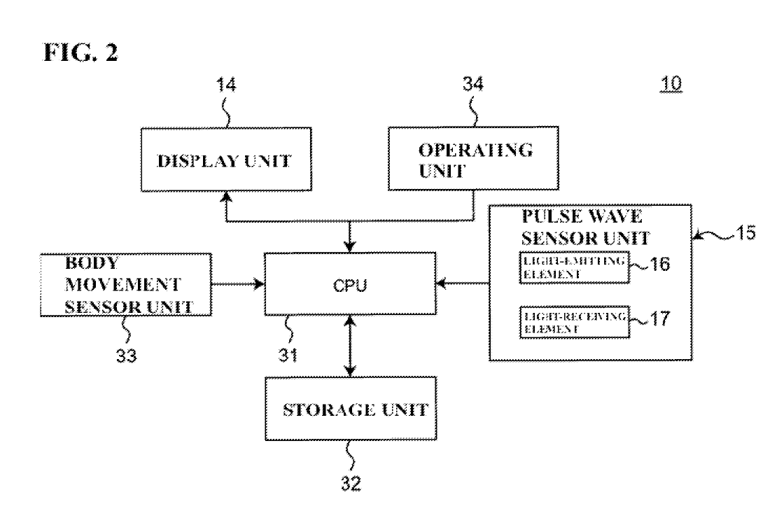

FIG. 3 illustrates an example of the circuit configuration of the pulse wave sensor unit 15 in the pulse measurement device 1. The pulse wave sensor unit 15 includes a pulse wave sensor controller 41 that controls operations of the pulse wave sensor unit 15 by operating under the control of the CPU 31.

The pulse wave sensor controller 41 drives the light-emitting element 16 in pulses by controlling a pulse driving circuit 42. In other words, the pulse driving circuit 42 controls a light emission state (frequency and duty) of the light-emitting element 16 by switching an NPN transistor based on a driving pulse supplied from the pulse wave sensor controller 41.

The pulse wave sensor controller 41 also controls the emitted light intensity (that is, a driving current) of the light-emitting element 16 by controlling an emitted light intensity control circuit 43. In other words, by changing the resistance value of a variable resistance based on an emitted light intensity control signal from the pulse wave sensor controller 41 controlled by the CPU 31, the emitted light intensity control circuit 43 controls the emitted light intensity of the light-emitting element 16 by driving the light-emitting element 16 with a driving current defined by that resistance value. That is, the emitted light intensity (the amount of light emitted, in other words) of the light-emitting element 16 increases as the driving current flowing in the light-emitting element 16 increases.

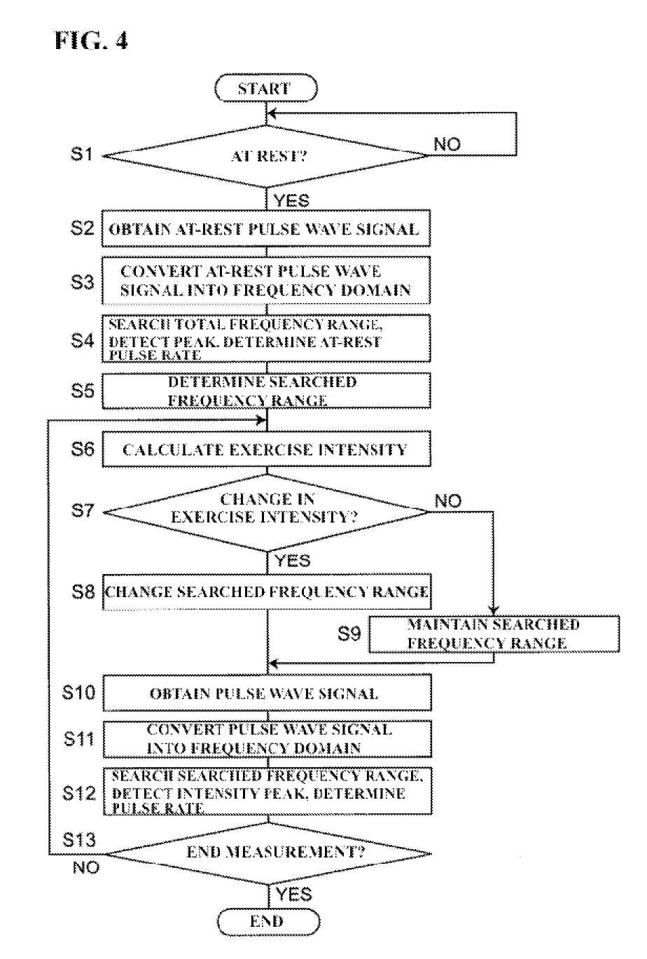

The light-receiving element 17 outputs a photoelectric output in accordance with the intensity of the received light. The pulse wave sensor controller 41 controls the light-emitting element 16 as described above, and controls a light receiving sensitivity (that is, a photoelectric output gain) of the light-receiving element 17 by controlling a light receiving sensitivity adjustment circuit 44. The light receiving sensitivity adjustment circuit 44 adjusts the magnitude of the photoelectric output from the light-receiving element 17 (a pulse wave DC component P_{DC} in FIG. 5A) by increasing/reducing the resistance value of the variable resistance in accordance with a photoelectric output control signal from the pulse wave sensor controller 41 controlled by the CPU 31.

Note that here, the photoelectric output from the light-receiving element 17 is referred to as the pulse wave DC component P_{DC}. Although the photoelectric output outputted from the light-receiving element 17 is actually a pulsating flow in which an AC component is superimposed over a constant level (a DC component), the magnitude of the pulsatory motion is extremely low compared to the magnitude of the photoelectric output, and thus the photoelectric output from the light-receiving element 17 is referred to here as the pulse wave DC component P_{DC}.

The photoelectric output from the light-receiving element 17 (the pulse wave DC component P_{DC} in FIG. 5A) is split into two branches, with one being inputted into a band pass filter (BPF) 45 and the other being inputted into an A/D conversion circuit (a DC component ADC) 47D.

The BPF 45 has a function for extracting the AC component from the photoelectric output P_{DC} outputted from the light-receiving element 17, and an amplifier 46 has a function for amplifying an output from the BPF 45. It is sufficient for the pass-band of the BPF 45 to contain a frequency band corresponding to a person's typical pulse rate range (30 BPM to 300 BPM) (that is, a frequency band of 0.5 Hz to 5 Hz). The AC component of the photoelectric output P_{DC} (a pulse wave AC component PS(t) in FIG. 5B) is outputted from the amplifier 46, and that output is inputted into an A/D conversion circuit (an AC component ADC) 47A.

The photoelectric signal P_{DC} outputted from the light-receiving element 17 is converted from an analog signal into a digital signal by the A/D converter 47D, and a digital signal corresponding to the pulse wave AC component PS(t) outputted from the ADC 47A is inputted into the CPU 31. The digital signal corresponding to the pulse wave AC component PS(t) is used to calculate the measurement subject's pulse rate, as will be described later. The photoelectric signal (pulse wave DC component P_{DC}) serving as the output from the ADC 47D is inputted into the CPU 31, and is used in a process for calculating parameters and the like for controlling the emitted light intensity.

Although the digital signals outputted from the ADC 47A (the AC component ADC) and the ADC 47D (the DC component ADC) are inputted into the CPU 31 in this example, the configuration may be such that the ADCs 47A and 47D are provided in the CPU 31.

The body movement sensor unit 33 includes an accelerometer 48. The accelerometer 48 measures the magnitude of acceleration acting on the measurement area and outputs a measurement result to an amplifier 49. The output of the amplifier 49 is inputted into an A/D conversion circuit (ADC) 50, and a digital signal containing acceleration information is inputted to the CPU 31 from the ADC 50. Here, the magnitude of the acceleration acting on the accelerometer 48 is considered to have a high correlation with the intensity of the measurement subject's exercise, and thus the output from the accelerometer 48 is used as an exercise intensity signal expressing the intensity of the measurement subject's exercise.

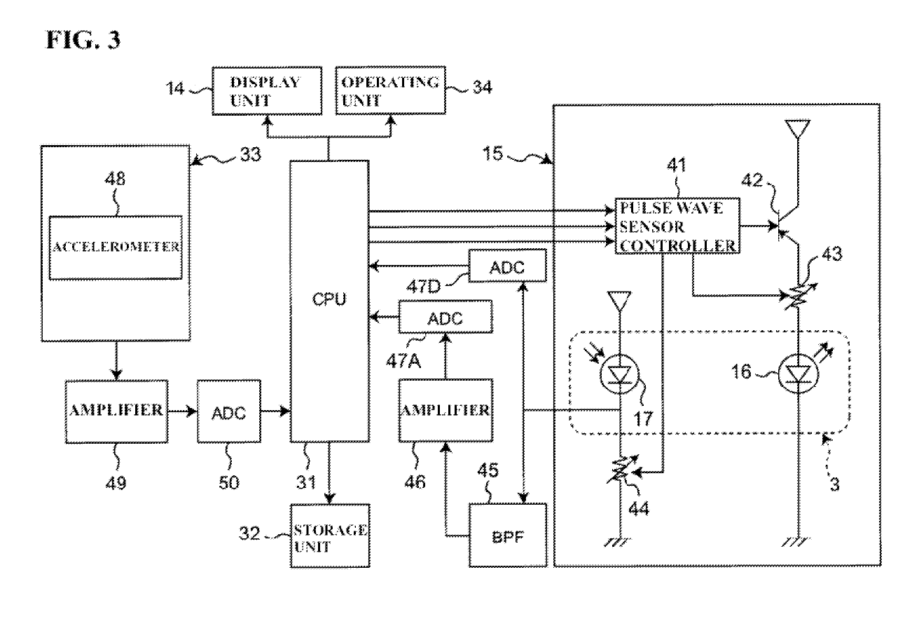

Overall, the pulse measurement device 1 operates according to the flow of a pulse measurement method, illustrated in FIG. 4.

To provide a general overview, first, when starting the measurement, the pulse measurement device 1 calculates the measurement subject's pulse rate while at rest (an at-rest pulse rate). Then, in the next measurement cycle, the pulse measurement device 1 determines, based on the at-rest pulse rate, a frequency range in which to search for a peak in the spectral intensity of the pulse wave signal (and more specifically, in the AC component of the pulse wave) expressed in the frequency domain (called a “searched frequency range”), extracts the peak in the spectral intensity present in the searched frequency range, and calculates the measurement subject's pulse rate based on the frequency of the extracted intensity peak. In the following measurement cycles, the pulse measurement device 1 shifts the searched frequency range from the searched frequency range used in the previous measurement based on the exercise intensity signal expressing the intensity of the measurement subject's exercise outputted from the body movement sensor unit 33, and by extracting a peak in the spectral intensity in that range, calculates the pulse rate in the present measurement cycle so as to track a change in the pulse rate from the pulse rate calculated in the previous measurement cycle.

i) First, as indicated in step S1, the CPU 31 determines whether or not the measurement subject is at rest based on the exercise intensity signal outputted from the body movement sensor unit 33, in order to measure the pulse rate while at rest. In the case where the CPU 31 determines that the measurement subject is at rest (“YES” in step S1), the process moves to step S2. When such is not the case, the CPU 31 repeats step S1 at a pre-set cycle. Note that in step S1, the CPU 31 may find a frequency spectrum of the pulse wave signal (the pulse wave AC component PS(t)) obtained from the pulse wave sensor unit and determine whether or not the measurement subject is at rest based on the shape of a spectral intensity distribution.

ii) Next, as indicated in step S2, the CPU 31 functions as a data obtainment unit that obtains, from the pulse wave sensor unit 15, the at-rest pulse wave signal (the pulse wave AC component PS(t)) expressing the measurement subject's pulse. More specifically, functioning as the data obtainment unit, the CPU 31 obtains the AC component PS(t) contained in the photoelectric signal P_{DC} (see FIGS. 5A and 5B).

FIG. 5A is a diagram illustrating an example of the photoelectric signal (the pulse wave DC component P_{DC}) outputted from the light-receiving element 17. In FIG. 5A, the horizontal axis represents time (in seconds), and the vertical axis represents the intensity of the pulse wave DC component P_{DC} (an arbitrary unit). The photoelectric signal (pulse wave DC component P_{DC}) is a pulsating flow containing a minute AC component, as described above. In other words, the pulse wave DC component P_{DC} is outputted as a pulsating flow in which a component (AC component) PS(t) that fluctuates cyclically along with the pulsatory motion of a body (the blood pulse wave, in other words) is superimposed on a constant level component (DC component), that does not fluctuate cyclically, produced by light absorbed and scattered by tissue, accumulated blood, or the like. Note that normally, the magnitude (amplitude) of the pulse wave AC component PS(t) that fluctuates cyclically is lower than the magnitude of the constant level component (the DC component) by approximately two digits. Accordingly, it is desirable to extract the pulse wave AC component PS(t) from the photoelectric signal (the pulse wave DC component P_{DC}) and amplify the pulse wave AC component PS(t) so that the component can be obtained as data. In this example, the amplifier 46 includes an op-amp, and an amplification gain of the pulse wave AC component is controlled by adjusting a resistivity between an input resistance and a feedback resistance under the control of the CPU 31. The pulse wave AC component PS(t) outputted from the amplifier 46 is converted into the pulse wave AC component PS(t), which is a digital signal, by the ADC 47A, and is inputted into the CPU 31.

FIG. 5B illustrates an example of a waveform of the pulse wave AC component PS(t) inputted into the CPU 31. Note that in FIG. 5B, the horizontal axis represents time (in seconds), and the vertical axis represents the intensity of the pulse wave AC component PS(t) (an arbitrary unit). The pulse wave AC component PS(t) changes cyclically in accordance with the pulsatory motion in the body (in other words, the pulse wave in the blood). In other words, the pulse wave AC component PS(t) is a pulse wave signal indicating the measurement subject's pulse. The pulse wave AC component PS(t) is stored in the storage unit 32 illustrated in FIG. 2, in time series.

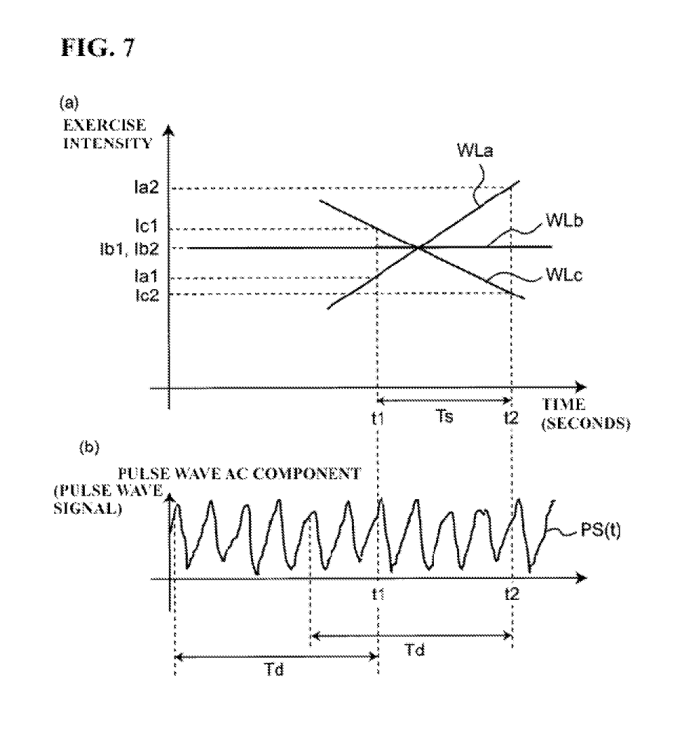

iii) Next, as indicated in step S3 of FIG. 4, the CPU 31 functions as a frequency conversion unit, converting the at-rest pulse wave signal (pulse wave AC component PS(t)) in the time domain, stored in the storage unit 32, into the frequency domain and finding a frequency spectrum (PS(f)) of the pulse wave signal (the pulse wave AC component PS(t)). More specifically, the CPU 31 functioning as the frequency conversion unit converts the at-rest pulse wave signal (the pulse wave AC component PS(t)) in the time domain, stored in the storage unit 32, into the frequency domain, and finds the frequency spectrum PS(f) of the pulse wave AC component while at rest. In this example, by functioning as the frequency conversion unit, the CPU 31 carries out a fast Fourier transform (FFT) on the at-rest pulse wave signal (pulse wave AC component PS(t)). As indicated by the example in FIG. 7B, the CPU 31 finds the frequency spectrum PS(f) of the at-rest AC component PS(t) contained in a period Td of a predetermined length (for example, 16 seconds, 8 seconds, 4 seconds, or the like) in the at-rest pulse wave AC component PS(t) stored in the storage unit 32 in time series.

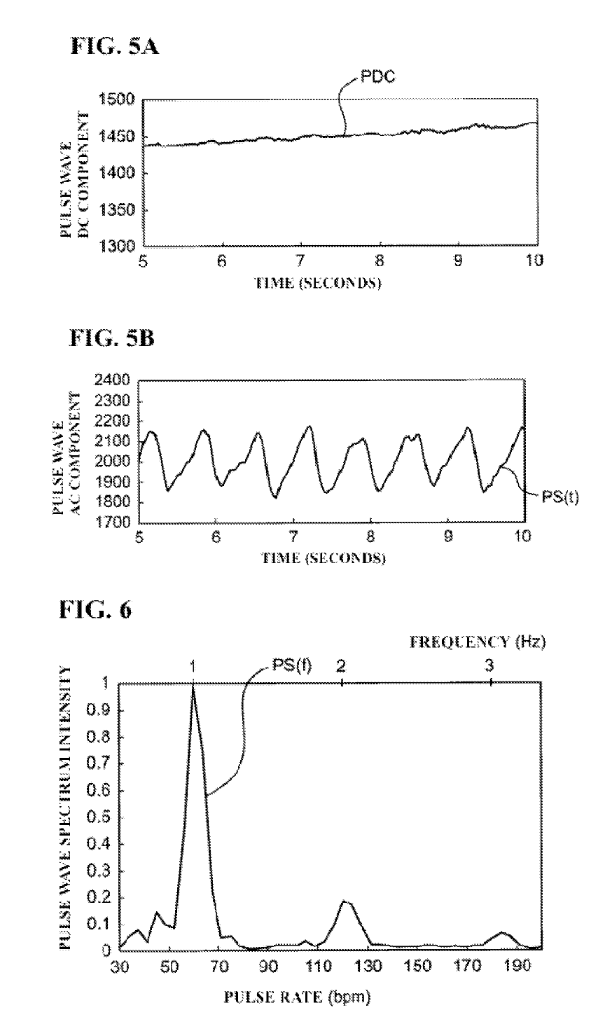

FIG. 6 is a diagram illustrating an example of the at-rest pulse wave AC component PS(f) converted into the frequency domain. In FIG. 6, the horizontal axis represents the pulse rate (in BPM (where 30 BPM corresponds to 0.5 Hz)), and the vertical axis represents the spectral intensity (an arbitrary unit). In this example, a large peak at approximately 60 BPM is seen in the at-rest AC component PS(f) converted into the frequency domain. A harmonic component thereof appears at approximately 120 BPM and approximately 180 BPM.

iv) Next, as indicated in step S4 of FIG. 4, the CPU 31 functions as a peak extraction unit, extracting an intensity peak in the searched frequency range set for the frequency spectrum. At the start of the measurement (in the case where the pulse rate of the measurement subject while at rest (the at-rest pulse rate) is to be found), the searched frequency range may be set to a total frequency range (for example, 30 BPM to 300 BPM, or in other words, 0.5 Hz to 5 Hz). In the example illustrated in FIG. 6, the CPU 31 extracts the intensity peak of the frequency spectrum PS(f) at approximately 60 BPM. With respect to the comparatively small intensity peaks at approximately 120 BPM and approximately 180 BPM, the CPU 31 takes those peaks as harmonic components of the intensity peak appearing at approximately 60 BPM, and discards those peaks. Next, the CPU 31 functions as a pulse rate calculation unit, finding the at-rest pulse rate of the measurement subject in accordance with the frequency of the extracted intensity peak, and determines that the at-rest pulse rate of the measurement subject is approximately 60 BPM based on the frequency of the extracted intensity peak (1 Hz, in the case of FIG. 6).

v) Next, as indicated in step S5 of FIG. 4, the CPU 31 functions as a searched range setting unit that sets the searched frequency range for searching for the intensity peak along the frequency axis of the aforementioned frequency spectrum. Specifically, the CPU 31 that functions as the searched range setting unit sets, as the searched frequency range for the next measurement cycle, a value within a predetermined ratio range (within plus/minus 20%, for example) relative to the pulse rate calculated in the previous measurement (here, the at-rest pulse rate (approximately 60 BPM)). For example, the CPU 31 sets a value range within plus/minus 20% of the pulse rate calculated in the previous measurement cycle (the at-rest pulse rate) as the searched frequency range for the next measurement cycle. When the pulse rate calculated in the previous measurement cycle is 60 BPM as shown in FIG. 6, a range from 48 BPM to 72 BPM is set as the searched frequency range for the next measurement cycle.

A processing loop from step S6 to step S13 in FIG. 4 carried out thereafter is a flow of processing for the second and subsequent pulse rate measurements counted from the start of measurement. The series of processes from step S6 to step S13 is executed each time the pulse rate is measured. This series of processes is carried out at a predetermined measurement cycle (at five-second intervals, for example (a time interval Ts indicated in FIG. 7)) until the measurement finishes. In the second and subsequent pulse rate measurements counted from the start of the measurement, the pulse measurement device 1 shifts the searched frequency range from the previous searched frequency range as necessary based on the exercise intensity signal outputted from the body movement sensor unit 33, extracts the spectral intensity peak in that searched frequency range, and calculates the pulse rate.

vi) As indicated in step S6 of FIG. 4, the CPU 31 functions as an exercise intensity obtainment unit, and obtains the exercise intensity signal, expressing the intensity of the measurement subject's exercise, from the body movement sensor unit 33.

vii) Next, as indicated in step S7, the CPU 31 that functions as the searched range setting unit compares the measurement subject's exercise intensity in the previous measurement cycle with the measurement subject's exercise intensity in the current measurement cycle based on the exercise intensity signal, and determines whether the exercise intensity in the current measurement cycle is greater than, equal to, or less than the exercise intensity in the previous measurement cycle.

FIG. 7(a) is a diagram illustrating a relationship between examples (three examples) of changes in the exercise intensity over time and measurement cycles. The horizontal axis represents time, and the vertical axis represents the intensity of the measurement subject's exercise determined based on the exercise intensity signal. Here, the exercise intensity may be an acceleration value outputted by the body movement sensor unit 33 (the accelerometer 48) at each time. Alternatively, the exercise intensity may be a value obtained by integrating the output of the accelerometer 48 over a predetermined time interval, or may be a value obtained by processing the exercise intensity signal outputted by the body movement sensor unit 33 through another predetermined calculation method. For example, the measurement subject's walking pitch (running pitch) outputted from the body movement sensor unit 33 (the accelerometer 48) may be found, and that pitch may be used as the exercise intensity.

A first exercise intensity time change example WLa is an example indicating a case where the exercise intensity in the current measurement cycle is greater than the exercise intensity in the previous measurement cycle. In the first exercise intensity time change example WLa, the exercise intensity in the previous measurement cycle (time t1) is la1, the exercise intensity in the current measurement cycle (time t2) is la2 (la2:la2>la1). In such a case, in step S7 of FIG. 4, the CPU 31 determines that the exercise intensity in the current cycle has changed so as to increase from the exercise intensity in the immediately-previous cycle (“YES” in step S7). Accordingly, the process moves to step S8.

A second exercise intensity time change example WLb is an example indicating a case where the exercise intensity in the current measurement cycle has not changed from the exercise intensity in the previous measurement cycle. In the second exercise intensity time change example WLb, the exercise intensity in the previous measurement cycle (time t1) is lb1, and the exercise intensity in the current measurement cycle (time t2) is lb2 (lb2:lb2=lb1). In such a case, in step S7 of FIG. 4, the CPU 31 determines that the exercise intensity in the current cycle has not changed from the exercise intensity in the immediately-previous cycle (“NO” in step S7). Accordingly, the process moves to step S9.

A third exercise intensity time change example WLc is an example indicating a case where the exercise intensity in the current measurement cycle is less than the exercise intensity in the previous measurement cycle. In the third exercise intensity time change example WLc, the exercise intensity in the previous measurement cycle (time t1) is lc1, and the exercise intensity in the current measurement cycle (time t2) is lc2 (lc2:lc2<lc1). In such a case, in step S7 of FIG. 4, the CPU 31 determines that the exercise intensity in the current cycle has changed so as to decrease from the exercise intensity in the immediately-previous cycle (“YES” in step S7). Accordingly, the process moves to step S8.

viii) As indicated in step S8 of FIG. 4, the CPU 31 functions as the searched range setting unit, and shifts the searched frequency range toward a higher frequency (a higher BPM) than the previous searched frequency range in the case where the exercise intensity in the current measurement cycle is greater than the exercise intensity in the previous measurement cycle (such as the case of the exercise intensity WLa in FIG. 7(a)).

Conversely, the CPU 31 shifts the searched frequency range toward a lower frequency (a lower BPM) than the previous searched frequency range in the case where the exercise intensity in the current measurement cycle is less than the exercise intensity in the previous measurement cycle (such as the case of the exercise intensity WLc in FIG. 7(a)).

As indicated in step S9, the CPU 31 does not shift the searched frequency range from the previous searched frequency range in the case where the exercise intensity in the current measurement cycle has not changed from the exercise intensity in the previous measurement cycle (such as the case of the exercise intensity WLb in FIG. 7(a)).

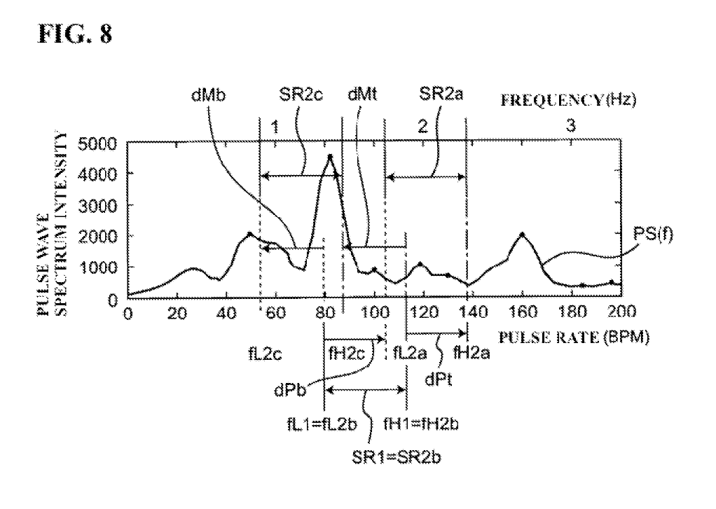

FIG. 8 is a diagram illustrating the searched frequency range being changed (or maintained) by step S8 and step S9 in FIG. 4. The horizontal axis represents the pulse rate (BPM), and the vertical axis represents the spectral intensity (an arbitrary unit). The frequency spectrum PS(f) illustrated in FIG. 8 corresponds to the pulse wave signal PS(t), from t=t2−Td to t=t2 in the pulse wave signal (the pulse wave AC component PS(t)) in the time domain shown in FIG. 7(b), being converted into the frequency domain.

In the example illustrated in FIG. 8, the searched frequency range in the previous measurement cycle is a frequency range SR1. The frequency range SR1 is a frequency range a spectral space defined by a lower limit frequency fL1 and an upper limit frequency fH1 (that is, fH1−fL1), and it is assumed that the intensity peak has been extracted from that range in the previous pulse measurement.

In step S8 of FIG. 4, in the case where the exercise intensity in the current measurement cycle is greater than the exercise intensity in the previous measurement cycle (such as the case of the exercise intensity WLa in FIG. 7(a)), the CPU 31 shifts the searched frequency range to a frequency range SR2a in higher frequencies (higher BPM) than the previous searched frequency range SR1. Through this, the searched frequency range in the current measurement cycle is a frequency range in a spectral space defined by a lower limit frequency fL2a (fL2a=fL1+dPb) and an upper limit frequency fH2a (fH2a=fH1+dPt) (that is, fH2a−fL2a). Here, dPb may be equal to dPt, and in this case, the spectral space of the current searched frequency range is the same as the spectral space of the previous searched frequency range. Meanwhile, the CPU 31 may increase the amount by which the searched frequency range is shifted (dPt and dPb in FIG. 8) the greater a difference between the exercise intensity in the current measurement cycle and the exercise intensity in the previous measurement cycle is (an exercise intensity difference in FIG. 7(a) (la2−la1)). Doing so makes it possible to track the pulse rate, which is likely to increase as the burden of exercise increases, with more certainty.

Conversely, the CPU 31 shifts the searched frequency range to a frequency range SR2c in lower frequencies (lower BPM) than the previous searched frequency range SR1 in the case where the exercise intensity in the current measurement cycle is less than the exercise intensity in the previous measurement cycle (such as the case of the exercise intensity WLc in FIG. 7(a)). Through this, the searched frequency range in the current measurement cycle is a frequency range in a spectral space defined by a lower limit frequency fL2c (fL2c=fL1−Mb) and an upper limit frequency fH2c (fH2c=fH1−dMt) (that is, fH2c−fL2c). Here, dMb may be equal to dMt, and in this case, the spectral space of the current searched frequency range is the same as the spectral space of the previous searched frequency range. Meanwhile, the CPU 31 may increase the amount by which the searched frequency range is shifted (dMt and dMb in FIG. 8) the greater a difference between the exercise intensity in the current measurement cycle and the exercise intensity in the previous measurement cycle is (an exercise intensity difference in FIGS. 7(a) (lc1−lc2)). Doing so makes it possible to track the pulse rate, which is likely to decrease as the burden of exercise decreases, with more certainty.

In step S9 of FIG. 4, the CPU 31 does not change the searched frequency range from the previous searched frequency range SR1 in the case where the exercise intensity in the current measurement cycle has not changed from the exercise intensity in the previous measurement cycle (such as the case of the exercise intensity WLb in FIG. 7(a)).

ix) As indicated in step S10 in FIG. 4, by functioning as the data obtainment unit, the CPU 31 obtains, from the storage unit 32, time series data of the pulse wave signal (the pulse wave AC component PS(t)) for the current measurement cycle. For example, in the case where time series data of a pulse wave signal (pulse wave AC component PS(t)) such as that shown in FIG. 7(b) is stored in the storage unit 32, the CPU 31 obtains, from the storage unit 32, the time series data of the pulse wave signal (the pulse wave AC component PS(t)) from time t2−Td to time t2 of the current measurement cycle.

x) Next, as indicated in step S11 of FIG. 4, by operating as the frequency conversion unit, the CPU 31 converts the time domain pulse wave signal (pulse wave AC component PS(t)) stored in the storage unit 32 into the frequency domain and finds the frequency spectrum (PS(f)) of the pulse wave signal (the pulse wave AC component PS(t)). For example, the CPU 31 carries out a fast Fourier transform (FFT) on the time series data of the pulse wave signal (the pulse wave AC component PS(t)) in a predetermined period Td obtained in step S10, and calculates the frequency spectrum PS(f) of the pulse wave signal as illustrated in FIG. 8.

xi) Then, as indicated in step S12 of FIG. 4, by functioning as the peak extraction unit, the CPU 31 extracts the intensity peaks (maximum points) of the frequency spectra in the searched frequency range of the current measurement cycle (SR2a, SR2b, or SR2c) set in step S8 or in step S9. Next, by functioning as the pulse rate calculation unit, the CPU 31 calculates the measurement subject's at-rest pulse rate based on the frequency at the extracted intensity peak.

xii) In step S13, the CPU 31 determines whether or not to end the pulse measurement, and in the case where the pulse measurement is to be continued, the process returns to step S6 and processing for the next measurement cycle is carried out.

As described thus far, the pulse measurement device 1 according to this preferred embodiment predicts a trend in fluctuations in the pulse based on the intensity of exercise of the measurement subject, and based on the direction of the predicted pulse fluctuation, shifts the previous searched frequency range toward higher frequencies or lower frequencies, maintains the same searched frequency range as the previous range, or the like, and extracts a spectral intensity peak produced by the pulse from the pulse wave signal in the frequency domain. By doing so, even in the case where, for example, an external disturbance component is superimposed on the pulse wave signal due to the measurement subject exercising, a spectral intensity peak produced by the external disturbance component will not be misrecognized as a spectral intensity peak caused by the pulse (or at least will be misrecognized less frequently), and thus the measurement subject's pulse rate can be measured correctly even when the measurement subject is not at rest.

The pulse measurement device 1 according to the preferred embodiment is a pulse measurement device that calculates a measurement subject's pulse rate based on a frequency spectral intensity distribution in a pulse wave signal obtained non-electrocardiographically. “Non-electrocardiographically” refers to a photoelectric system, for example, but is not limited thereto. A piezoelectric system and the like are also included in non-electrocardiographic methods, in addition to photoelectric systems.

The pulse measurement device 1 according to the preferred embodiment extracts and uses, as the pulse wave signal, a component, in the photoelectric output P_{DC}, that fluctuates in a cycle within a range estimated to be the pulse rate of a measurement subject (30 BPM to 300 BPM). However, the photoelectric output P_{DC} may be used directly as the pulse wave signal.

The aforementioned pulse measurement method may be constructed as a program for causing a computer to execute the method.

Such a program (a pulse measurement program) may be recorded on a computer-readable recording medium, and made distributable in such a form. By installing the pulse measurement program in a generic computer, the aforementioned pulse measurement method can be executed by the generic computer.

In addition, a program stored in the storage unit 32 may be encoded on a memory or other non-transitory computer-readable recording medium (a memory, a hard disk drive, an optical disk, or the like), and a generic computer may then be caused to execute the aforementioned pulse measurement method. The program may also be distributed over the Internet or the like.

Although the CPU 31 carries out a fast Fourier transform (FFT) as the conversion into the frequency domain in the aforementioned example, the invention is not limited thereto. Any other conversion method may be employed as long as the method is capable of converting the photoelectric signal P_{DC} in the time domain into the frequency domain.

Furthermore, a dedicated hardware logic circuit that executes the aforementioned pulse measurement method may be used as the CPU 31. In other words, at least one of the data obtainment unit, the exercise intensity obtainment unit, the searched range setting unit, the peak extraction unit, and the pulse rate calculation unit may be realized as dedicated hardware circuitry.

In addition, in the aforementioned example, when it is determined in step S1 of FIG. 4 that the measurement subject is at rest, in step S4 of FIG. 4, a frequency indicating the maximum intensity peak contained in the frequency spectrum of the pulse wave signal is found as the measurement subject's at-rest pulse rate. However, the invention is not limited thereto. The measurement subject's at-rest pulse rate may be found by counting the number of peaks or valleys in the pulse wave signal (the pulse wave AC component PS(t)) and finding the number of fluctuations per minute based on a number of repetitions in the fluctuation of the pulse wave signal (the pulse wave AC component PS(t)).

The aforementioned preferred embodiments are merely examples, and many variations thereon can be carried out without departing from the scope of this invention.

While preferred embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A pulse measurement device comprising:
a data obtainment unit configured to obtain a pulse wave signal expressing a pulse by detecting a pulse wave of a measurement subject using a pulse wave sensor;
an exercise intensity obtainment unit configured to obtain an exercise intensity signal expressing an intensity of exercise performed by the measurement subject by detecting movement in the measurement subject using a body movement sensor;
a storage unit configured to store the pulse wave signal;
a frequency conversion unit configured to find a frequency spectrum of the pulse wave signal by converting the time-domain pulse wave signal stored in the storage unit into a frequency domain;
a searched range setting unit configured to set a searched frequency range for searching for an intensity peak along a frequency axis of the frequency spectrum;
a peak extraction unit configured to extract an intensity peak from the frequency spectrum in the set searched frequency range; and
a pulse rate calculation unit configured to find a pulse rate of the measurement subject based on a frequency of the extracted intensity peak,
wherein the searched range setting unit changes the searched frequency range based on an exercise intensity indicated by the exercise intensity signal.

2. The pulse measurement device according to claim 1,
wherein the frequency conversion unit, the exercise intensity obtainment unit, the searched range setting unit, the peak extraction unit, and the pulse rate calculation unit repeat the aforementioned process at a predetermined cycle; and
in the case where the pulse rate calculation unit has calculated a first value as the pulse rate of the measurement subject in a first cycle, the searched range setting unit sets a value present in a predetermined ratio range relative to the first value as the searched frequency range for a second cycle that follows the first cycle.

3. The pulse measurement device according to claim 1,
wherein in the case where an exercise intensity obtained by the exercise intensity obtainment unit in a fourth cycle that follows a third cycle is greater than an exercise intensity obtained in the third cycle, the searched range setting unit sets a frequency range shifted toward a higher frequency than the searched frequency range for the third cycle as the searched frequency range for the fourth cycle.

4. The pulse measurement device according to claim 3,
wherein the searched range setting unit sets the searched frequency range for the fourth cycle so as to have the same spectral space as the searched frequency range for the third cycle.

5. A method for measuring a pulse rate of a measurement subject carried out by a pulse measurement device, the method comprising:
a data obtainment step of obtaining a pulse wave signal expressing a pulse of the measurement subject using a pulse wave sensor;
a storage step of storing the pulse wave signal in a storage unit;
a frequency conversion step of finding a frequency spectrum of the pulse wave signal by converting the time-domain pulse wave signal stored in the storage unit into a frequency domain;
an exercise intensity obtainment step of obtaining an exercise intensity signal expressing an intensity of exercise performed by the measurement subject using a body movement sensor;
a searched range setting step of setting a searched frequency range for searching for an intensity peak along a frequency axis of the frequency spectrum;
a peak extraction step of extracting an intensity peak from the frequency spectrum in the set searched frequency range; and
a pulse rate calculation step of finding the pulse rate of the measurement subject based on a frequency of the extracted intensity peak,
wherein the searched range setting step includes a step of changing the searched frequency range based on an exercise intensity indicated by the exercise intensity signal.

6. A pulse rate measurement computer program capable of causing a computer of a pulse measurement device to execute:
a data obtainment step of obtaining a pulse wave signal expressing a pulse of a measurement subject using a pulse wave sensor;
a storage step of storing the pulse wave signal in a storage unit;
a frequency conversion step of finding a frequency spectrum of the pulse wave signal by converting the time-domain pulse wave signal stored in the storage unit into a frequency domain;
an exercise intensity obtainment step of obtaining an exercise intensity signal expressing an intensity of exercise performed by the measurement subject using a body movement sensor;
a searched range setting step of setting a searched frequency range for searching for an intensity peak along a frequency axis of the frequency spectrum;
a peak extraction step of extracting an intensity peak from the frequency spectrum in the set searched frequency range; and
a pulse rate calculation step of finding the pulse rate of the measurement subject based on a frequency of the extracted intensity peak,
wherein the searched range setting step includes a step of changing the searched frequency range based on an exercise intensity indicated by the exercise intensity signal.
